(19) United States
(12) Patent Application Publication
(10) Pub. No.: US 2007/0172429 A1
(43) Pub. Date: Jul. 26, 2007

(54) Labeling compositions and methods of use for deterrent trackability

(76) Inventors: Xiaolian Gao, Houston, TX (US); David M. Bear, Wellesley, MA (US); George M. Church, Brookline, MA (US); Xiaochuan Zhou, Houston, TX (US)

Correspondence Address: SUTHERLAND ASBILL & BRENNAN LLP, 999 PEACHTREE STREET, N.E., ATLANTA, GA, 30309, US

(21) Appl. No.: 11/464,361

(22) Filed: Aug. 14, 2006

Related U.S. Application Data
(60) Provisional application No. 60/707,600, filed on Aug. 12, 2005.
(60) Provisional application No. 60/711,213, filed on Aug. 25, 2005.
(60) Provisional application No. 60/728,981, filed on Oct. 21, 2005.

(51) Int. Cl. A61K 9/44; C12Q 1/68; G01N 33/551

(52) U.S. Cl. 424/10.2; 435/6; 436/524; 977/924

(57) ABSTRACT

Compositions and methods for controlling and tracking items are provided. More particularly, labeling compositions useful as a coding system to deter diversion of items of medication or other valued items are provided, wherein the labeling compositions comprise at least 25 unique identifiers, wherein the at least 25 unique identifiers are selected from a set of at least 50 unique identifiers. Also provided are methods for using such compositions to provide trackability throughout a chain of custody.

### CROSS REFERENCE TO RELATED APPLICATIONS

This application claims the priority benefit of U.S. Provisional Application No. 60/707,600, filed Aug. 12, 2005; U.S. Provisional Application No. 60/711,213, filed Aug. 25, 2005; and U.S. Provisional Application No. 60/728,981, filed Oct. 21, 2005. These applications are incorporated herein in their entirety by reference.

### BACKGROUND OF THE INVENTION

This invention is generally in the field of compositions and methods for controlling and tracking items. More particularly, the invention includes labeling compositions useful as a coding system to deter diversion of items of medication or other valued items, and methods for using such compositions to provide trackability throughout a chain of custody.

The treatment of patients in health care settings typically involves the use of various medical items such as prescription drugs and medications, nonprescription drugs and medications, medical and surgical supplies, and consumable medical equipment. To serve the needs of the patients, sufficient stocks of such medical items must be kept available for use. Because such items are likely the subject of unauthorized diversion, it is important for a health care provider to be able to accurately control and track the use of such items.

The unauthorized diversion of legal medications from the supply chain from pharmaceutical manufacturer to the patient is a significant problem. In many cases, the diversion occurs when a patient obtains a prescription for a drug, purchases the drug, and resells the drug to another individual or into an illegal network where the drug is sold and ultimately is consumed by an end user. Examples of legal medications that are often diverted include prescription drugs such as analgesic drugs (e.g., NSAIDs and narcotics), sedatives (e.g., benzodiazepines), and psychostimulants (e.g., amphetamines and amphetamine-like drugs). These drugs have great value when sold by patients, but they also have severe morbidity and even mortality when abused. For this reason, it is highly desirable to provide methods and compositions for the tracking of such legal medications in order to deter the unauthorized diversion of such legal medications.

Presently, control of drugs prescribed to patients is only occasionally performed and is conducted by calling the patient in and checking whether he has the correct number of untaken pills. This control method is time consuming for both the pharmacy or clinic and the patient, and the control method can be easily circumvented if the patient merely buys the correct number of pills on the street and presents them to the proper authority. Because the present control methods are ineffective, better methods for tracking medications and deterring their unauthorized diversion are needed.

Moreover, when an unauthorized person is found with a prescribed drug, only that person bears liability for prosecution. There is no straightforward check on the original seller of the drug. At present, most pharmacies, clinics, or other health care settings have no means for identifying that a drug is from a particular prescription that was provided to a specific person. Therefore, even if the person found with the drug gives the authorities the name of the person who supplied the drug, a time-consuming and difficult process is needed to prove beyond a reasonable doubt that the illegal transaction occurred. Accordingly, the present control methods do not adequately deter the diversion of drugs. It would be desirable to provide an identification means such that a sample of a medication can be analyzed to definitively determine the source of the medication. In particular, it would be desirable to determine the specific individual prescription that was filled by a pharmacy, clinic, or other health care setting. It also would be desirable to provide methods to deter the diversion of a particular medication from its prescribed use.

The treatment of patients in pharmacies, clinics, and other health care settings also typically involves other valued items that are likely to be the subject of unauthorized diversion such as, for example, certificates of value and currency. Accordingly, it also would be highly desirable to provide labeling means and tracking methods for such other valued items.

### SUMMARY OF THE INVENTION

The present compositions comprise a carrier particle, and a labeling mixture associated with the carrier particle, wherein the labeling mixture comprises a plurality of unique identifiers. In a preferred embodiment, the labeling mixture comprises at least 25 unique identifiers, wherein the at least 25 unique identifiers are selected from a set of at least 50 unique identifiers. In some embodiments, the at least 25 unique identifiers are associated with a single carrier particle. In other embodiments, each of the at least 25 unique identifiers is associated with a different carrier particle and the composition comprises at least 25 carrier particles. In certain embodiments, the carrier particle is a ferrite bead. The identifiers may be selected from the group consisting of an oligonucleotide, a peptide, an oligoglycan, a dye, a pharmaceutically acceptable excipient, a mass tag, and combinations thereof. In certain embodiments, the unique identifier is an oligonucleotide that comprises a central coding region flanked by two amplification regions. In certain embodiments, the central coding region comprises about 5-10 nucleotides, and each amplification region comprises about 15-30 nucleotides.

In other embodiments, the present compositions for use in labeling an item comprise a labeling mixture that comprises a plurality of unique identifiers. In certain embodiments, the item is a medication. In a preferred embodiment, the labeling mixture comprises at least 25 identifiers, wherein the at least 25 unique identifiers are selected from a set of at least 50 unique identifiers.

Also described are pharmaceutical dosage forms associated with a labeling composition, wherein the labeling composition comprises a labeling mixture that comprises a plurality of unique identifiers, and wherein the labeling mixture is either associated directly with the pharmaceutical dosage form or is indirectly associated with the pharmaceutical dosage form via a carrier particle. In a preferred embodiment, the labeling mixture comprises at least 25 unique identifiers, wherein the at least 25 unique identifiers are selected from a set of at least 50 unique identifiers. In one embodiment, the labeling composition is associated with a solid oral dosage form by dusting the dosage form with the labeling composition. In certain embodiments, the labeling mixture comprises a plurality of unique identifiers associated with a carrier particle and wherein the carrier particle is a ferrite bead. The unique identifiers of the labeling composition are selected from the group consisting of an oligonucleotide, a peptide, an oligoglycan, a dye, a pharmaceutically acceptable excipient, a carrier particle, a mass tag, and combinations thereof.

The present methods for labeling an item comprise the steps of: a) providing an item; b) associating the item with a labeling mixture that comprises a combination of at least 25 unique identifiers, wherein the at least 25 unique identifiers are selected from a set of at least 50 unique identifiers, and wherein the labeling mixture is either associated directly with the item or is indirectly associated with the pharmaceutical dosage form via a carrier particle; and c) documenting the combination of at least 25 unique identifiers associated with the item. In certain embodiments, the methods are used to label an item selected from the group consisting of a pill, a dermal patch, a medical device, a certificate of value, and currency. In one embodiment, the labeling composition is associated with the item by dusting the item with the labeling composition. In certain embodiments, the carrier particle of the labeling composition is a ferrite bead. The unique identifiers of the labeling composition are selected from the group consisting of an oligonucleotide, a peptide, an oligoglycan, a dye, a pharmaceutically acceptable excipient, a carrier particle, a mass tag, and combinations thereof. In certain embodiments, the identifier is an oligonucleotide that comprises a central coding region flanked by two amplification regions. In certain embodiments, the central coding region comprises about 5-10 nucleotides, and each amplification region comprises about 15-30 nucleotides.

The present methods may be used for tracking items that have been labeled as described herein. In a preferred embodiment, the present methods comprise the steps of: a) obtaining an item which may have been labeled with an associated labeling mixture; b) determining the combination of at least 25 identifiers associated with the item, if present; and c) comparing the determined combination with documentation to identify the source of the item. In certain embodiments, the methods for determining the at least twenty-five identifiers associated with an item involve the use of an epifluorescence microscope, a spectrophotometer, a Coulter counter, or a combination thereof.

### BRIEF DESCRIPTION OF THE FIGURES

FIG. 1 is a cross-sectional view of one embodiment of a pharmaceutical dosage form as described herein. Panel A depicts a cross-sectional view of a solid oral dosage form that has been dusted with the present labeling composition. Panel B depicts a cross-sectional view of one embodiment of a single coded entity of the labeling composition.

FIG. 2 is a schematic representation of the parallel synthesis of the present oligonucleotide or peptide identifiers.

FIG. 3 is a schematic representation of the present methods for labeling an item (left column) and for identifying a labeled item (right column).

### DESCRIPTION OF THE INVENTION

This invention fulfills in part the need in the art to identify new, unique compositions and methods for the labeling and tracking of items. The disclosed compositions and methods are particularly useful for the labeling and tracking of prescription medications to deter their unauthorized diversion.

The present compositions and methods implement a factorial coding system to deter diversion of items of medication or other valued items by providing trackability throughout a chain of custody. In certain embodiments, a set of n identifiers (preferably at least 50) are provided, thus providing a plurality of unique mixtures that are m member subsets (n/2) of the set of n identifiers, and then using individual subsets of the set of n identifiers to uniquely mark an object or substance. The object or substance can then be tracked by suitable methods known in the art of molecular biology. This factorial design provides a manufacturing strategy, for example, by sampling the identifiers from n containers and creating probes in advance to recognize each of the n identifiers. The factorial design and selection of identifiers also defines the number of potential codes available since each of the identifiers must be one of the n identifiers in the set. For this reason, there is some “error correction” possible. For example, given a set of 50 identifiers, a particular code will consist of 25 identifiers. If only 24 of the 25 identifiers are able to be detected from the code on a particular item, it is known that the undetected identifier must be one of 26.

Unless otherwise noted, the terms used herein are to be understood according to conventional usage by those of ordinary skill in the relevant art. In addition to the definitions of terms provided below, definitions of common terms in molecular biology may also be found in Rieger et al., 1991, Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “an oligonucleotide” can mean that at least one oligonucleotide can be utilized. Furthermore, as used herein, the terms “comprise,” “comprising,” “include,” and “including” are intended to be open, non-limiting terms, unless the contrary is expressly indicated.

The present invention may be understood more readily by reference to the following detailed description of the preferred embodiments of the invention and the Examples included herein. However, before the present compositions and methods are disclosed and described, it is to be understood that this invention is not limited to specific nucleic acids, specific peptides, or specific methods, etc., as such may, of course, vary, and the numerous modifications and variations therein will be apparent to those skilled in the art.

Standard techniques for cloning, DNA isolation, amplification, and purification, for enzymatic reactions involving DNA ligase, DNA polymerase, restriction endonucleases and the like, and various separation techniques are those known and commonly employed by those skilled in the art. Certain techniques are provided herein in more detail. In addition, a number of standard techniques are described in Sambrook et al., 1989, Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

The present compositions and methods may be used to aid in the control of items, and in particular medical items, such as prescription medications. In one embodiment, each batch of medication required for a single prescription is physically associated with a unique identifying code. As used herein, the term “code” refers to a combination of unique identifiers that have been used to specifically label a particular item such as the medication in a particular prescription. As used herein, the term “associated with” means that the relevant molecules or substances adhere to, are bonded to, or are electrostatically attached to one another, for example. As also used herein with respect to a medication, the term “associated with” means that the code is contained in the medication if it is a liquid, gel, or in tablet, caplet, or capsule form, or is on or near the surface of each tablet, caplet, or capsule. In various embodiments, the term “pharmaceutical dosage form” refers to a solution, suspension, emulsion, tablet, pill, pellet, capsule, capsule containing liquid, powder, or sustained-release formulation, suppository, aerosol, sprays, suspensions, or any other form suitable for use. As used herein, the term “solid oral dosage form” refers to a tablet, caplet, pill, pellet, capsule, or powder formulation. The unit dosage form may be encapsulated, e.g., with soluble or bioerodible polymeric films to control release. The pharmaceutically active ingredient may be in the form of microparticles or nanoparticles, which themselves may include a controlled release (coating or matrix) material or structure known in the art. In one embodiment, the pharmaceutically acceptable vehicle is a capsule (See, e.g., U.S. Pat. No. 5,698,155). Other examples of suitable pharmaceutical vehicles are described in Remington's Pharmaceutical Sciences, Alfonso R. Gennaro ed., Mack Publishing Co. Easton, Pa., 19th ed., 1995, pp. 1447 to 1676, which is incorporated herein by reference.

If coded entities are admixed with the medication, a sufficient number of coded entities are admixed that a portion of the prescription which might be sold contains at least one entity. As used herein, the terms “coded entity” and “coding entity” refer to a molecule from the labeling composition that contains the unique identifying code.

The coded entities may be sequestered in indigestible microspheres or other packaging that allows them to pass through the digestive tract without absorption and remain readable. Microsphere encapsulation of medication is well known in the art, particularly with respect to the timed release of medication where the dissolution rate of the encapsulation is controlled.

The medication may be associated with the coded entity in the factory which manufactures the medication, allowing the cost of encoding to be spread over many prescriptions. Alternatively, the medication may be associated with the coded entity at the distribution point to the wholesalers or retail outlets, such as pharmacies, clinics, and other health care settings.

In one embodiment, a pill is associated with the coded entity by providing a prebarcoded dispensing bottle that is supplied with a powder that comprises coded entities, wherein the unique identifying code carried by the coded entities corresponds to the barcode present on the dispensing bottle. In some instances, a “foreign” pill (i.e. a pill that is not part of the prescription associated with that unique identifying code) might be later dropped into the bottle and shaken to take on the appropriate code. To prevent this later labeling of a foreign pill, the powder with coded entities may be provided on a plicated cylindrical sleeve which would be placed in the dispensing bottles supplied to the pharmacy, clinic, or other health care setting. The bottle with barcode label and sleeve would represent the “code installer.” After pouring pills from the count tray into the dispensing bottle, the pharmacist would close the dispensing bottle, shake well, and then open the bottle to remove the coding sleeve. The powder with coding entities should adhere well to the pills during that initial exposure; the “bonding” with the bottle should occur at dispensing; and there should be virtually no free powder left in the bottle if pills were added by the patient or by a third party after the initial dispensing of the prescription by the pharmacy, clinic, or other health care setting. Similarly, blisterpacks also could be pre-filled with the powder comprising coded entities, and the blisterpacks also could carry a barcode that corresponds to the unique identifying code carried by the coded entities. In another embodiment, a pre-barcoded container may be supplied with a cap, along with a packet of sticky to viscous liquid coding powder attached to the bottom (inside of the container), and a central pinhole opening in top of the packet. Pills may be poured into the container, the cap replaced, and the packet pierced through the cap with a pin. After shaking well, the cap may be removed and replaced with a dispensing cap. The container then has the barcode of the powder now associated with the pills. In another embodiment, the coding powder is contained in the cap and is released into the container upon twisting the cap on to the container.

Alternatively, the pills could be combined with the powder prior to placing in a prebarcoded dispensing bottle, wherein the unique identifying code carried by the coded entities corresponds to the barcode present on the dispensing bottle. In such embodiments, the powder could be provided in a separate container in which the pills could be added and shaken to coat them with the powder before transferring to the provided prebarcoded dispensing bottle.

The present labeling compositions comprise a carrier particle, and a labeling mixture associated with the carrier particle, wherein the labeling mixture comprises a plurality of unique identifiers. As used herein with respect to the labeling mixture, the term “associated with” means that the plurality of unique identifiers within the labeling mixture adhere to, are bonded to, are electrostatically attached to, or are otherwise physically attached to the carrier particle. In certain embodiments, the carrier particle is a ferrite bead or another biocompatible material in particulate form.

As used herein, the term “carrier particle” or “microparticle” includes microspheres and microcapsules, as well as microparticles, unless otherwise specified and refers to any such molecules that are generally recognized as safe (GRAS). Microparticles may or may not be spherical in shape. Microparticles can be rod like, sphere like, acicular (slender, needle-like particle of similar width and thickness), columnar (long, thin particle with a width and thickness that are greater than those of an acicular particle), flake (thin, flat particle of similar length and width), plate (flat particle of similar length and width but with greater thickness than flakes), lath (long, thin, blade-like particle), equant (particles of similar length, width, and thickness, this includes both cubical and spherical particles), lamellar (stacked plates), or disc like. Microcapsules are defined as microparticles having an outer shell surrounding a core of another material, for example, a pharmaceutical agent. The core can be gas, liquid, gel, or solid. Microspheres can be solid spheres, can be porous and include a sponge-like or honeycomb structure formed by pores or voids in a matrix material or shell, or can include a single internal void in a matrix material or shell.

The Shell Material

In some embodiments, the agent microparticles include a shell material. The shell material can be a synthetic material or a natural material. The shell material can be water soluble or water insoluble. The microparticles can be formed of non-biodegradable or biodegradable materials. Examples of types of shell materials include polymers, amino acids, sugars, proteins, carbohydrates, and lipids. Polymeric shell materials can be degradable or non-degradable, erodible or non-erodible, natural or synthetic.

Representative synthetic polymers include poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxyethyl cellulose, cellulose triacetate, and cellulose sulphate sodium salt jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof. As used herein, “derivatives” include polymers having substitutions, additions of chemical groups, for example, alkyl, alkylene, hydroxylations, oxidations, and other modifications routinely made by those skilled in the art.

Examples of biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof. Examples of natural polymers include proteins such as albumin and prolamines, for example, zein, and polysaccharides such as alginate, cellulose and polyhydroxyalkanoates, for example, polyhydroxybutyrate. Examples of preferred non-biodegradable polymers include ethylene vinyl acetate, poly(meth)acrylic acid, polyamides, copolymers and mixtures thereof.

Bioadhesive polymers can be of particular interest. Examples of these include polyanhydrides, polyacrylic acid, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Representative amino acids that can be used in the shell include both naturally occurring and non-naturally occurring amino acids. The amino acids can be hydrophobic or hydrophilic and may be D amino acids, L amino acids or racemic mixtures.

The shell material can be the same or different from the excipient material, if present. In one embodiment, the excipient can comprise the same classes or types of material used to form the shell. In another embodiment, the excipient comprises one or more materials different from the shell material. In this latter embodiment, the excipient can be a surfactant, wetting agent, salt, bulking agent, etc.

Excipients

The term “excipient” refers to any non-active ingredient of the formulation intended to facilitate delivery and administration by the intended route. For example, the excipient can comprise proteins, amino acids, sugars or other carbohydrates, starches, lipids, or combinations thereof. The excipient may enhance handling, stability, aerodynamic properties, and dispersibility of the active agent.

The excipient is a dry powder (e.g., in the form of microparticles,) which may be blended with drug microparticles. In one embodiment, the excipient microparticles have a volume average size between about 10 and 500 μm, preferably between 20 and 200 μm, more preferably between 40 and 100 μm.

Examples of excipients include pharmaceutically acceptable carriers and bulking agents, including sugars such as lactose, mannitol, trehalose, xylitol, sorbitol, erythritol, dextran, sucrose, and fructose. These sugars may also serve as wetting agents. Other suitable excipients include surface active agents, dispersants, osmotic agents, binders, disintegrants, glidants, diluents, color agents, flavoring agents, sweeteners, and lubricants. Examples include sodium desoxycholate; sodium dodecylsulfate; polyoxyethylene sorbitan fatty acid esters, e.g., polyoxyethylene 20 sorbitan monolaurate (TWEEN™ 20), polyoxyethylene 4 sorbitan monolaurate (TWEEN™ 21), polyoxyethylene 20 sorbitan monopalmitate (TWEEN™ 40), polyoxyethylene 20 sorbitan monooleate (TWEEN™ 80); polyoxyethylene alkyl ethers, e.g., polyoxyethylene 4 lauryl ether (BRIJ™ 30), polyoxyethylene 23 lauryl ether (BRIJ™ 35), polyoxyethylene 10 oleyl ether (BRIJ™ 97); polyoxyethylene glycol esters, e.g., poloxyethylene 8 stearate (MYRJ™ 45), poloxyethylene 40 stearate (MYRJ™ 52); Tyloxapol; Spans; and mixtures thereof.

Examples of binders include starch, gelatin, sugars, gums, polyethylene glycol, ethylcellulose, waxes and polyvinylpyrrolidone. Examples of disintegrants (including super disintegrants) includes starch, clay, celluloses, croscarmelose, crospovidone and sodium starch glycolate. Examples of glidants include colloidal silicon dioxide and talc. Examples of diluents include dicalcium phosphate, calcium sulfate, lactose, cellulose, kaolin, mannitol, sodium chloride, dry starch and powdered sugar. Examples of lubricants include talc, magnesium stearate, calcium stearate, stearic acid, hydrogenated vegetable oils, and polyethylene glycol.

In another embodiment, the excipient comprises binders, disintegrants, glidants, diluents, color agents, flavoring agents, sweeteners, lubricants, or combinations thereof for use in a solid oral dosage form. Examples of solid oral dosage forms include capsules, standard tablets, orally disintegrating tablets and wafers.

As used herein, microparticles are particles having a size of 0.5 to 1000 microns. The microparticles preferably have a number average diameter of between 0.5 μm and 5 mm. The microparticles can be made using a variety of techniques known in the art. Suitable techniques include solvent precipitation, crystallization, spray drying, melt extrusion, compression molding, fluid bed drying, solvent extraction, hot melt encapsulation, phase inversion encapsulation, and solvent evaporation.

As used herein, the term “labeling mixture” refers to the coding powder, liquid, or other substance that comprises the coding entities or unique identifiers. In certain embodiments, the labeling mixture includes coding entities that each comprise at least 15 unique identifiers, more preferably at least 20 unique identifiers, and most preferably at least 25 unique identifiers. As used herein, the term “unique identifiers” refers to the molecules that are used to create a code for labeling an item as described herein. The identifiers are selected from the group consisting of an oligonucleotide, a peptide, an oligoglycan, a dye, a pharmaceutically acceptable excipient, a carrier particle, a mass tag, and combinations thereof. As used herein in the context of its use as an identifier, the term “pharmaceutically acceptable excipient” refers to any excipient that is approved by a regulatory agency of the Federal or a state government or listed in the U.S. Pharmacopeia or other generally recognized pharmacopeia for use in animals, mammals, and more particularly in humans, and that is not already being used in the preparation of a particular medication. In certain embodiments, the pharmaceutically acceptable excipient is selected from the group consisting of lactose, mannitol, corn starch, potato starch, crystalline cellulose, a cellulose derivative, acacia, a gelatin, sodium carboxymethylcellulose, talc, magnesium carbonate, magnesium stearate, glucose, sucrose, sorbitol, malt, rice, flour, chalk, silica gel, sodium saccharin, sodium stearate, glycerol stearate, glycerol monostearate, sodium chloride, dried skim milk, propylene, glycol, ethanol, water, keratin, colloidal silica, and urea.

As also used herein, the terms “colorant” and “dye” are intended to include color pigment formulations that are approved by a regulatory agency of the Federal or a state government for use in animals, mammals, and more particularly in humans. For example, numerous formulations, including newly available pearlescent pigments are available through Colorcon (http://www.colorcon.com/pharma/index.html).

As used herein, the term “peptide” is intended to include molecules consisting of two or more amino acids. Dipeptides (molecules consisting of two amino acids) may be formed by methods well known in the art to create a set of, for example, 64 unique peptide identifiers. The amino acids of the peptide identifiers are preferably selected to be easily resolved by thin layer chromatography or by an automated Sanger technique or other methods known in the art. The dipeptide may be eluted from the bead and read by a sequencer. Thirty-two peptide identifiers may be associated with a single carrier particle. Alternatively, multiple copies of one of the thirty-two peptide identifiers may be associated with each carrier particle to make a coding powder composed of thirty-two carrier particles, each with a different peptide identifier.

In certain preferred embodiments, the identifier is an oligonucleotide. The present compositions and methods implement an oligonucleotide coding system to deter diversion of items of medication or other valued items by providing trackability throughout a chain of custody. Generally, in certain embodiments, a set of n oligonucleotides (for example, by generating a library), providing a plurality of unique mixtures that are m member subsets of the set of n nucleotides, and then using individual subsets of the set of n oligonucleotides to uniquely mark an object or substance. The object or substance can then be tracked by suitable methods known in the art of molecular biology. Short oligonucleotides can be produced rapidly and inexpensively in large quantities using good manufacturing practices well known in the art (See, e.g., examples herein; see also http://usa.eurogentec.com/code/en/page_{—}08_{—}459_{—}0.htm, and http://www.oligosetc.com/profile.php). For example, as discussed in more detail below in the Examples, a moderate sized library can be created containing 64 highly purified oligonucleotides (e.g., each oligonucleotide being 46 nucleotides in length and having a 6 base coding region flanked by 20 base primers for PCR amplification). Then distinctive mixtures can be produced, e.g., based on unique combinations of subsets of 32 of these molecules. In this way, a very large number of unique mixes (e.g., approximately 1.8^{E18} for this example) can be created.

A particular mix can be assigned a number which is expressed in a variety of ways, e.g., by a 64 bit binary number in which 1 indicates the presence of an index oligo, 0 its absence, represented in a bar code (1 or 2 dimensional) or associated with the packing of an aliquot of the mix available for application. By dusting or other methods of associating small quantities of a specific mix with an item or its immediate surroundings, those in control of the item can mark it uniquely, so that authorities can track items subject to misuse or theft, such as pills, dermal patches, currency surrendered under duress or taken from a mix-dusted environment.

The present oligonucleotide identifiers comprise a central coding region flanked by two amplification regions. In certain embodiments, the central coding region comprises about 5-10 nucleotides, and each amplification region comprises about 15-30 nucleotides. In one embodiment, the central coding region consists of 6 nucleotides, and the amplification regions consist of 20 nucleotides. As used herein, the term “central coding region” refers to the variable sequence of 5-10 nucleotides that provide the code for the unique identifier. As also used herein, the term “amplification region” refers to the 15-30 nucleotide invariable region of the identifier that may be used for hybridization with a primer for the amplification of the identifier or that may be used for hybridization with a probe that has been labeled, for example, with a fluorophore. In other preferred embodiments, the chemistry of the oligonucleotide identifiers are chosen to be resistant to nuclease and proteolytic activity, with high affinity for the target sequence with low affinity for nonspecific binding. For example, an oligonucleotide identifier can be chemically synthesized using naturally occurring nucleotides or variously modified nucleotides designed to increase the biological stability of the molecules or to increase the physical stability of the duplex formed between the complimentary nucleic acids, e.g., phosphorothioate derivatives and acridine substituted nucleotides can be used. Preferred examples of classes of modified nucleotides which can be used to generate the nucleic acid probes are a 2′-O-methyl nucleotide and a peptide nucleic acid backbone. Additional examples of modified nucleotides which can be used to generate the nucleic acid probes include, for example, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In some embodiments, the unique identifiers may be a combination of an oligonucleotide, a peptide, an oligoglycan, a dye, and/or different shapes, sizes, and color of carrier particles. Depending on the type of identifiers used, the identifiers may be detected by use of, for example, antisense DNA, spectrophotometry, flurocytometry, and computerized shape detection, among other techniques well known in the art. For example, with respect to the use of carrier particles as the unique identifiers, 5 different shapes of particles may be used with 5 different colors or hues of particles and 2 sizes of particles to create a set of 50 unique identifiers (based on combinations of the given shapes, colors, and sizes) from which unique sets of 25 identifiers may be produced. Particle size analysis can be performed on a Coulter counter, by light microscopy, scanning electron microscopy, transmission electron microscopy, laser diffraction methods, light scattering methods or time of flight methods. Where a Coulter counter method is described, the powder is dispersed in an electrolyte, and the resulting suspension analyzed using a Coulter Multisizer II fitted with a 50-μm aperture tube. Where a laser diffraction method is used, the powder is dispersed in an aqueous medium and analyzed using a Coulter LS230, with refractive index values appropriately chosen for the material being tested. Color and hue analysis may be performed on a spectrophotometer at varying wavelengths to distinguish between the possible colors and hues.

The present labeling compositions may comprise any factorial combination of molecules, including but not limited to, organic combinatorial syntheses that can be used as identifying mixtures added to drugs and later decoded and/or quantitated by mass sprectrometry. Examples of mass tags are disclosed in Xu et al., (1997) “Electrophore Mass Tag Dideoxy DNA Sequencing,” Analytical Chemistry, 69: 3595-3602; Shchepinov et al., 1999, “Trityl mass-tags for encoding in combinatorial oligonucleotide synthesis,” Nucleic Acids Symp Ser., (42): 107-8; Hwang et al., 2004, “OBOC Small-Molecule Combinatorial Library Encoded by Halogenated Mass-Tags,” Org. Lett., 6(21):3829-3832; and Arlinghaus et al., 1997, “Multiplexed DNA sequencing and diagnostics by hybridization with enriched stable isotope labels,” Anal. Chem., 69(8): 1510-7.

Also described are pharmaceutical dosage forms associated with a labeling composition, wherein the labeling composition comprises a carrier particle and a labeling mixture associated with the carrier particle, wherein the labeling mixture comprises a plurality of unique identifiers. In one embodiment, the labeling composition is associated with a solid oral dosage form by dusting the dosage form with the labeling composition. In certain embodiments, the carrier particle of the labeling composition is a ferrite bead. The unique identifiers of the labeling composition may be selected from the group consisting of an oligonucleotide, a peptide, an oligoglycan, a dye, a pharmaceutically acceptable excipient, a carrier particle, a mass tag, and combinations thereof.

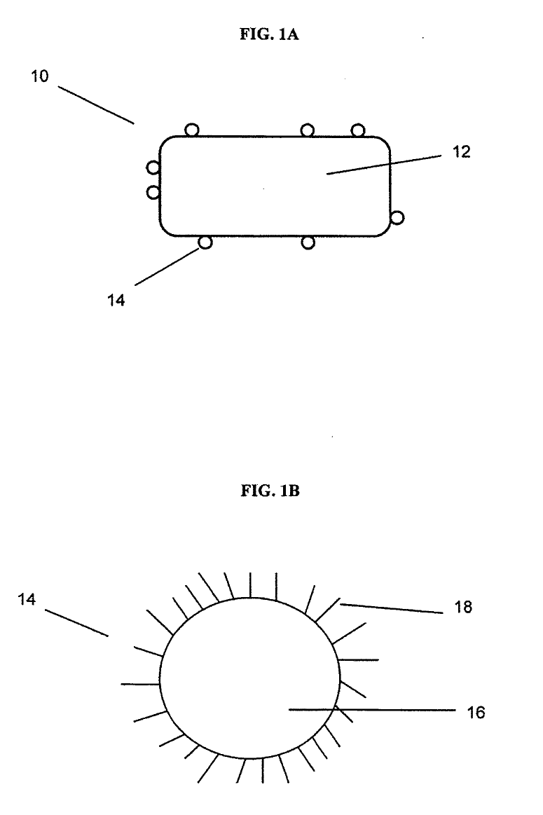

FIG. 1A illustrates a labeled dosage form (e.g., capsule, pill, or tablet) 10 which includes the solid oral dosage form 12 with labeled carrier particles 14 attached to the solid oral dosage form 12. FIG. 1B illustrates a labeled carrier particle 14 which includes twenty-five unique identifiers 18 attached to a single carrier particle 16.

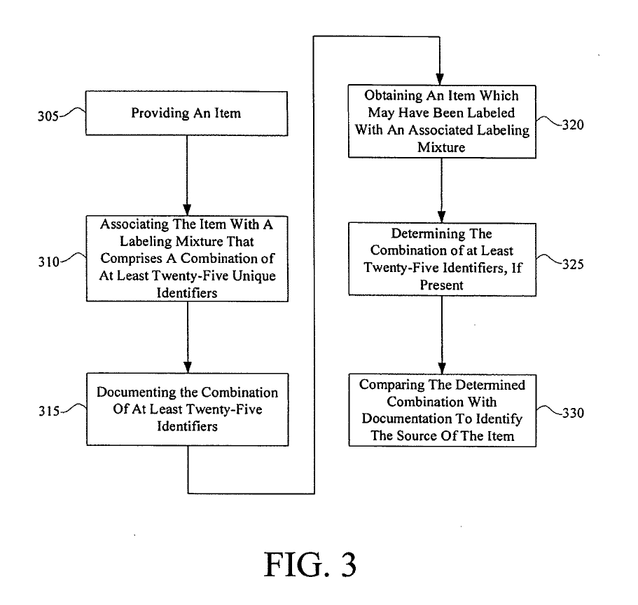

The present methods use the labeling compositions described herein, such that diversion of a medication or other valued item can be traced back to the original supplier of the medication or item. The present methods for labeling an item are depicted in the left column of FIG. 3 and comprise the steps of: a) providing an item (305); b) associating the item with a labeling mixture that comprises a combination of at least 25 unique identifiers (310), wherein the at least 25 unique identifiers are selected from a set of at least 50 unique identifiers, and wherein the labeling mixture is either associated directly with the item or is indirectly associated with the item via a carrier particle; and c) documenting the combination of at least 25 unique identifiers associated with the item (315). As used herein, the term “associated directly with” means that the unique identifiers in the labeling mixture adhere to, are bonded to, or are electrostatically attached to the item being labeled. As also used herein, the term “indirectly associated with” means that the unique identifiers in the labeling mixture adhere to, are bonded to, or are electrostatically attached to a carrier particle which is then adhered to, bonded to, or electrostatically attached to the item being labeled. In certain embodiments, the methods are used to label an item selected from the group consisting of a pill, a dermal patch, a medical device, a certificate of value, and currency. As used herein, the term “dermal patch” refers to a product for the transdermal administration of a substance. For example, the patch may comprise an impermeable outer layer, a peelable protective later, and a matrix which contains an active ingredient or substance, or a reservoir which contains the active ingredient or substance and comprises a semipermeable membrane. As used herein, the term “medical device” refers to an instrument, apparatus, implement, machine, contrivance, implant, in vitro reagent, or other similar or related article, including a component part, or accessory which is intended for use in the diagnosis of disease or other conditions, or in the cure, mitigation, treatment, or prevention of disease, in man or other animals, or intended to affect the structure or any function of the body of man or other animals. For example, this term includes, among other things, simple items (e.g., tongue depressors and bedpans), but also includes complex equipment (e.g., pacemakers and laser surgical devices) and in vitro diagnostic products (e.g., lab equipment, reagents, and test kits). As also used herein, the term “currency” refers to money in any form when in actual use as a medium of exchange. As used herein, the term “certificate of value” refers to a certificate that may be used as a medium of exchange (e.g., a gift certificate).

In one embodiment, the labeling composition is associated with a medication by admixing the labeling composition with the medication during the manufacture of the medication. In another embodiment, the labeling composition is associated with the item by dusting the item with the labeling composition at the time of distribution to the retailer. In yet another embodiment, the labeling composition is associated with the item by dusting the item with the labeling composition at the time of distribution to a patient. As used herein, the term “dusting” includes coating an item with a powder comprising the coded entities as disclosed in this application.

In certain embodiments of these methods, the carrier particle of the labeling composition is a ferrite bead. The unique identifiers of the labeling composition may be selected from the group consisting of an oligonucleotide, a peptide, an oligoglycan, a dye, a pharmaceutically acceptable excipient, a carrier particle, a mass tag, and combinations thereof. In certain embodiments, the identifier is an oligonucleotide that comprises a central coding region flanked by two amplification regions. In certain embodiments, the central coding region comprises about 5-10 nucleotides, and each amplification region comprises about 15-30 nucleotides.

The present methods are useful for tracking items that have been labeled as described herein. For example, a medication desirably can be traced to the individual prescription filled by a pharmacy, clinic, or other health care setting which is delivered to a named individual. The present compositions and methods enable the production, delivery, and use of a medication whereby the diversion of the medication from its prescribed use may be monitored. In a preferred embodiment, the methods are shown in the right hand column of FIG. 3 and comprise the steps of: a) obtaining an item which may have been labeled with an associated labeling mixture (320); b) determining the combination of at least 25 unique identifiers associated with the item, if present (325); and c) comparing the determined combination with documentation to identify the source of the item (330). In certain embodiments, the methods for determining the at least twenty-five identifiers associated with an item involve the use of an epifluorescence microscope, a spectrophotometer, a Coulter counter, or a combination thereof. In other embodiments, the methods for determining the identifiers involve PCR amplification and/or sequencing methods that are well known in the art.

For example, a pharmacy receiving the medication from the medication supplier will record the unique identifying code along with, for example, a prescription number (the prescription number is or can be associated with the patient), the physician supplying the prescription, the date, etc. If the medication is diverted from the intended use, the unique identifying code is read from a recovered item, the prescription data is determined, and appropriate action is taken to punish the patient responsible for the diversion and/or to prevent further occurrences of such diversion.

In another use, packages of pills carrying a unique oligonucleotide mix may be numbered by any of the methods above and logged to an individual at the time of dispensing, discouraging illicit diversion by virtue of traceability and implicating individuals who have handled the labeled pills. An individual may be given a card containing an identifying oligonucleotide mix, which could be carried or kept at a pharmacy, and on dispensing medication, a very small quantity of the mix may be transferred from the card and deposited on pills or inside their packaging. Detection of the mix on pills, hands, or within, for example, an underground laboratory producing drugs of abuse such as methamphetamine, can be facilitated, e.g., by PCR amplification (as the oligonucleotides having been selected for uniform replication), hybridization technology, and/or sequencing methods well known in the art of molecular biology.

In order to handle the case that a forensic sample contains dust from multiple pills, the labeling composition (“dust”) can be formulated such that many coded entities (and therefore, many molecules of each oligonucleotide) are associated with each dust particle. For example, a one micron bit of dust (the size of a bacterial cell) can hold millions of copies of each oligonucleotide assuming mixes described above. In practice, hundreds of copies should be adequate for detection. For routine practice, several dust particles would be analyzed by conventional PCR methods. If that analysis proves the dust particles to be a mixture, then individual dust particles would be analyzed, e.g., most cost-effectively in separate tubes, by multiplex polony sequencing, for example, as follows.

For practice of multiplex polony sequencing, the dust particles must be stable in water at room temperature (such that DNA from different particles do not mix), yet capable of releasing the DNA or making it accessible to DNA polymerase upon change of temperature or pH or exposure to enzymes and/or detergents. The particles also should not aggregate excessively in water. The particles are diluted at a density such that they can be easily distinguished optically with an epifluorescence microscope as described herein.

A device that can mix and dispense mixtures of oligonucleotides, such as those described above, could consist of n containers from which m wells are sampled under computer control with disposable (or highly sterilized) pipette tips to make a mixed solution, which is then dried and powdered by standard procedures. For example, the oligonucleotides could be created to have double-biotin on their 5′ ends and, following mixing, avidin-coated one micron magnetic beads could be added, binding up to one million oligonucleotides molecules of all types in the mix. As an alternative, the process could be carried out manually as only 32 pipette steps are required and there are redundant checks.

The present compositions and methods are applicable to any item that is likely to be the subject of unauthorized diversion, and particularly applicable to medications. Examples of medications that are likely to be diverted include, but are not limited to, analgesic drugs (e.g. NSAIDs and narcotics), sedatives (e.g. benzodiazepines), and psychostimulants (e.g. amphetamines and amphetamine-like drugs).

Examples of benzodiazepines include, but are not limited to, alprazolam, bromazepam, chlordiazepoxide, clobazam, clonazepam, clorazepate, diazepam, estazolam, flunitrazepam, flurazepam, halazepam, ketazolam, loprazolam, lorazepam, lormetazepam, medazepam, midazolam, nitrazepam, nordazepam, oxazepam, prazepam, quazepam, temazepam, tetrazepam, triazolam, and DMCM.

Examples of opioid agonists include, but are not limited to, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, tramadol, pharmaceutically acceptable salts thereof, and mixtures thereof.

Examples of non-opioid analgesics include, but are not limited to, non-steroidal anti-inflammatory agents, such as aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam, and pharmaceutically acceptable salts thereof, and mixtures thereof. Other suitable non-opioid analgesics include the following, non-limiting, chemical classes of analgesic, antipyretic, nonsteroidal anti-inflammatory drugs: salicylic acid derivatives, including aspirin, sodium salicylate, choline magnesium trisalicylate, salsalate, diflunisal, salicylsalicylic acid, sulfasalazine, and olsalazin; para-aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicarns (piroxicam, tenoxicam), and pyrazolidinediones (phenylbutazone, oxyphenthartazone); and alkanones, including nabumetone. For a more detailed description of the NSAIDs, see Paul A. Insel, Analgesic-Antipyretic and Anti-inflammatory Agents and Drugs Employed in the Treatment of Gout, in Goodman & Gilman's The Pharmacological Basis of Therapeutics 617-57 (Perry B. Molinhoff and Raymond W. Ruddon eds., 9.sup.th ed 1996) and Glen R. Hanson, Analgesic, Antipyretic and Anti-Inflammatory Drugs in Remington: The Science and Practice of Pharmacy Vol II 1196-1221 (A. R. Gennaro ed. 19th ed. 1995) which are hereby incorporated by reference in their entireties.

Examples of psychostimulants include, but are not limited to, methylphenidate, amphetamine, dextroamphetamine, PCP, DXM, PMA, ketamine, caffeine, amphetamine, methamphetamine, ephedrine, pseudoephedrine, aspirin, paracetamol, and fentanyl.

### EXAMPLES

### Example 1

Methods of Producing Labeling Compositions with Unique Oligonucleotide Identifiers

Parallel Synthesis of Oligonucleotide Identifiers

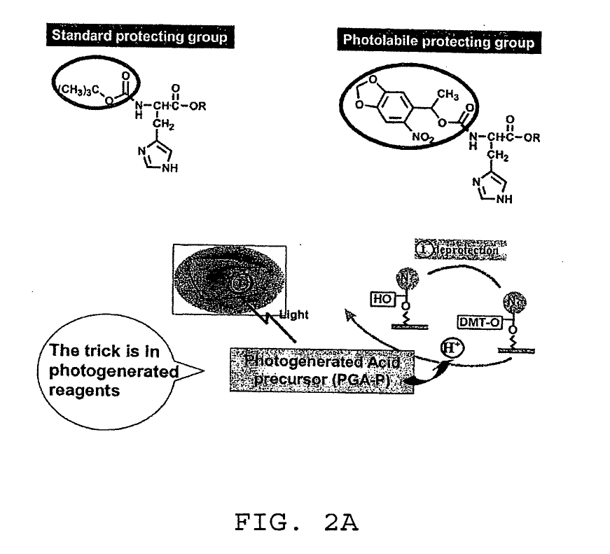

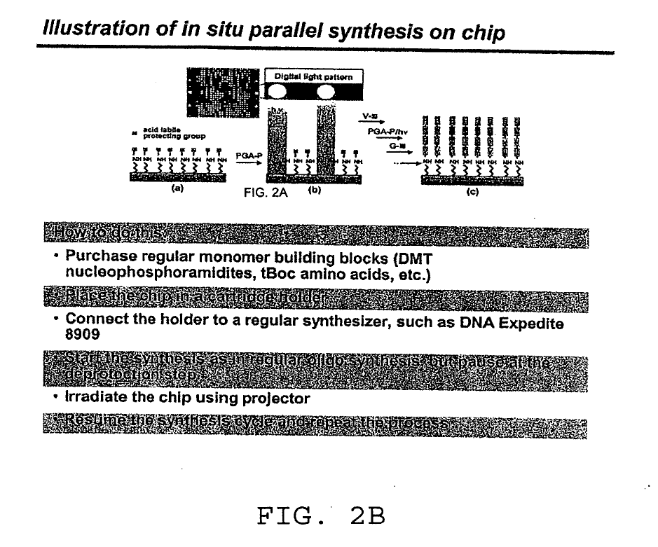

The present oligonucleotide identifiers comprise a central coding region that comprises about 5-10 nucleotides that is flanked by two amplification regions that comprise about 15-30 nucleotides. These oligonucleotides can be produced directly on a functional surface. For reference, see, for example, Zhou et al., 2004, “Microfluidic PicoArray synthesis of oligodeoxynucleotides and simultaneous assembling of multiple DNA sequences,” Nuc. Acids Res. 32(18):5409-5417. Regular monomer building blocks such as DMT nucleophosporamidites and tboc amino acids may be purchased commercially. A chip is placed in a cartridge holder, and the assembly is connected to a regular DNA synthesizer (e.g. DNA Expedite 8909). Synthesis is started as in regular oligonucleotide synthesis, but is paused at the deprotection step. During that step, photogenerated acid (PGA) is used to deprotect the 4′,4-dimethoxytrityl (DMT) group at selected reaction sites. The chip is irradiated using a digital photolithographic projector; synthesis is resumed; and this cycle is repeated (FIG. 2).

PicoArray synthesis provides an excellent means to make thousands and tens of thousands of oligonucleotides at the cost and time of preparing only a few oligonucleotides by conventional methods. Accordingly, this method of synthesis is particularly useful for producing the present oligonucleotide identifiers. However, traditional methods of synthesis well known in the art also could be used to synthesize the oligonucleotides as well. In addition, synthesis of unique peptide identifiers is also possible using the parallel synthesis techniques.

### Example 2

Analysis of Labeling Compositions with Unique Oligonucleotide Identifiers

Example of Analyzing Two Beads in a Mixture:

Ferrite beads, or other carrier particles, comprising 32 types of oligonucleotide identifiers (i.e. 46 nucleotide oligonucleotides; “46-mers”) are produced by methods known in the art, for example as described in Example 1. The oligonucleotide identifiers comprise a variable central region consisting of a 6 nucleotide combination (“tag” or “code”), flanked on each side by 20 nucleotide regions for amplification and/or hybridization. The tags may be selected from the following set of 64 tags:

| | | | |
|---|---|---|---|
| AAAAAA | (SEQ ID NO:1) | AACAAC | (SEQ ID NO:2) |
| AAGAAG | (SEQ ID NO:3) | AATAAT | (SEQ ID NO:4) |
| ACAACA | (SEQ ID NO:5) | ACCACC | (SEQ ID NO:6) |
| ACGACG | (SEQ ID NO:7) | ACTACT | (SEQ ID NO:8) |
| AGAAGA | (SEQ ID NO:9) | AGCAGC | (SEQ ID NO:10) |
| AGGAGG | (SEQ ID NO:11) | AGTAGT | (SEQ ID NO:12) |
| ATAATA | (SEQ ID NO:13) | ATCATC | (SEQ ID NO:14) |
| ATGATG | (SEQ ID NO:15) | ATTATT | (SEQ ID NO:16) |
| CAACAA | (SEQ ID NO:17) | CACCAC | (SEQ ID NO:18) |
| CAGCAG | (SEQ ID NO:19) | CATCAT | (SEQ ID NO:20) |
| CCACCA | (SEQ ID NO:21) | CCCCCC | (SEQ ID NO:22) |
| CCGCCG | (SEQ ID NO:22) | CCTCCT | (SEQ ID NO:23) |
| CGACGA | (SEQ ID NO:24) | CGCCGC | (SEQ ID NO:25) |
| CGCCGC | (SEQ ID NO:26) | CGTCGT | (SEQ ID NO:27) |
| CTACTA | (SEQ ID NO:28) | CTCCTC | (SEQ ID NO:29) |
| CTGCTG | (SEQ ID NO:30) | CTTCTT | (SEQ ID NO:31) |
| GAAGAA | (SEQ ID NO:32) | GACGAC | (SEQ ID NO:33) |
| GAGGAG | (SEQ ID NO:35) | GATGAT | (SEQ ID NO:36) |
| GCAGCA | (SEQ ID NO:37) | GCCGCC | (SEQ ID NO:38) |
| GCGGCG | (SEQ ID NO:39) | GCTGCT | (SEQ ID NO:40) |
| GGAGGA | (SEQ ID NO:41) | GGCGGC | (SEQ ID NO:42) |
| GGGGGG | (SEQ ID NO:43) | GGTGGT | (SEQ ID NO:44) |
| GTAGTA | (SEQ ID NO:45) | GTCGTC | (SEQ ID NO:46) |
| GTGGTG | (SEQ ID NO:47) | GTTGTT | (SEQ ID NO:48) |
| TAATAA | (SEQ ID NO:49) | TACTAC | (SEQ ID NO:50) |
| TAGTAG | (SEQ ID NO:51) | TATTAT | (SEQ ID NO:52) |
| TCATCA | (SEQ ID NO:53) | TCCTCC | (SEQ ID NO:54) |
| TCGTCG | (SEQ ID NO:55) | TCTTCT | (SEQ ID NO:56) |
| TGATGA | (SEQ ID NO:57) | TGCTGC | (SEQ ID NO:58) |
| TGGTGG | (SEQ ID NO:59) | TGTTGT | (SEQ ID NO:60) |
| TTATTA | (SEQ ID NO:61) | TTCTTC | (SEQ ID NO:62) |
| TTGTTG | (SEQ ID NO:63) | TTTTTT | (SEQ ID NO:64) |

The number of unique carrier particles that may be produced from this set of 64 tags is 64!/(32!32!), which is 1.8E18 (where 64!=64*63*62* . . . 4*3*2).

The unique carrier particles, each comprising 32 oligonucleotide identifiers can be distinguished from one another through standard molecular analysis. For example, if a dust is obtained that includes a mixture of two bead types, these beads can both be identified as depicted in the following illustration.

Bead Type A is: 1111111111111111111111111111111100000000000000000000000000000000, which holds the first 32 oligonucleotide identifiers (46-mers).

Bead Type B is: 1010101010101010101010101010101010101010101010101010101010101010, which holds the odd numbered 46-mers.

The carrier particles or beads with attached oligonucleotide identifiers under analysis are immobilized as described in Shendure et al., 2005, Science Express (e.g. as single-stranded 46-mers 5′ biotin immobilized to streptavidin beads themselves immobilized by a gel). Then 64 binding reactions are performed using 9 nucleotide (“9-mers”) probes which contain 6 nucleotides from the left (i.e. 5′ on the upper strand) of the tag plus the leftmost 3 bases of the tag. These probes are paired with 9-mers for the right sides.

So, for example, if the tag of the first two oligonucleotide types on bead A read: 5′-gtacgtAAAAAAgcgcgc-3′ (SEQ ID NO:65) and 5′-gtacgtAACAACgcgcgc-3′ (SEQ ID NO:66), then the corresponding Probe Pair #1 would be: right-5′-gcgcgcTTT-3′ (SEQ ID NO:67) and left-5′-TTTacgtac-3′ (SEQ ID NO:68). Probe Pair #2 would be: right-5′-gcgcgcGTT-3′ (SEQ ID NO:69) and left-5′-GTTacgtac-3′ (SEQ ID NO:70). Beads of type A, but not type B, would react positively with pair #2 because type B beads do not include the second oligonucleotide type. That is, the 18-mer formed by ligation would be resistant to washing, and the beads of type A would fluoresce more brightly if either 9-mer of the pair has a covalently attached fluorophore.

Alternatively, a generic right 20-mer (e.g. 5′-gccgatcgaatgagagcgcgc-3′ (SEQ ID NO:71)) could be used, and 50 fluorescently labeled left 9-mers could be tested serially (e.g. #1: 5′-TTTTTTacg-3′ (SEQ ID NO:72), #2: 5′-GTTGTTacg-3′ (SEQ ID NO:73), etc). Type A and type B beads would result in strong binding with probe #1; and only A beads would bind probe #2 strongly (i.e. the probe would hybridize to the bead and be resistant to washing). The right generic 20-mer is shown below in bold on the lower strand, along with the probe #2 that is regular font on the lower strand. The probes are shown on the lower strand where they would hybridize to the Bead A oligonucleotide #2 to form a double stranded 29-mer oligonucleotide.

| Bead A oligo #2: | |
|---|---|
| 5′-gtacgtAACAACgcgcgcctctctaagctagcc | (SEQ ID NO:74) |
| g-3′ | |
| 3′gcaTTGTTGcgcgcggagagattcgatcggc- | (SEQ ID NO:75) |
| 5′ | |

As a variation on the above, four 9-mer oligonucleotides, each with a distinct fluorophore, could be used simultaneously and measured with appropriate filters. The ratio of the two pill types can be assessed by the ratio of the bead types determined by the hybridization assays described above.

Publications cited herein are incorporated by reference. Modifications and variations of the methods and devices described herein will be obvious to those skilled in the art from the foregoing detailed description. Such modifications and variations are intended to come within the scope of the appended claims.

We claim:

1. A composition comprising,
a) at least one carrier particle, and
b) a labeling mixture associated with the at least one carrier particle, wherein the labeling mixture comprises a plurality of unique identifiers.

2. The composition of claim 1, wherein the labeling mixture comprises at least 25 unique identifiers, and wherein the at least 25 unique identifiers are selected from a set of at least 50 unique identifiers.

3. The composition of claim 2, wherein the at least 25 unique identifiers are associated with a single carrier particle.

4. The composition of claim 2, wherein each of the at least 25 unique identifiers is associated with a single carrier particle, and wherein the composition comprises at least 25 carrier particles.

5. The composition of claim 2, wherein the carrier particle is a ferrite bead.

6. The composition of claim 2, wherein the identifier is selected from the group consisting of an oligonucleotide, a pharmaceutically acceptable excipient, a dye, a peptide, an oligoglycan, a mass tag, and combinations thereof.

7. The composition of claim 6, wherein the unique identifier comprises an oligonucleotide.

8. The composition of claim 7, wherein the oligonucleotide comprises a central coding region flanked by two amplification regions.

9. The composition of claim 8, wherein the central coding region comprises about 5-10 nucleotides and wherein each amplification region comprises about 15-30 nucleotides.

10. The composition of claim 6, wherein the identifier is a dye selected from the group consisting of a colorant and a fluorescent dye.

11. The composition of claim 6, wherein the identifier is a peptide.

12. The composition of claim 6, wherein the identifier is an oligoglycan.

13. A composition for use in labeling a medication, comprising a labeling mixture that comprises a plurality of unique identifiers.

14. The composition of claim 13, wherein the labeling mixture comprises at least 25 unique identifiers, and wherein the at least 25 unique identifiers are selected from a set of at least 50 unique identifiers.

15. The composition of claim 13, wherein the unique identifiers are selected from the group consisting of an oligonucleotide, a carrier particle, a pharmaceutically acceptable excipient, a dye, a peptide, an oligoglycan, a mass tag, and combinations thereof.

16. A pharmaceutical dosage form associated with a labeling composition, wherein the labeling composition comprises a labeling mixture that comprises a plurality of unique identifiers, wherein the labeling mixture is either associated directly with the pharmaceutical dosage form or is indirectly associated with the pharmaceutical dosage form via a carrier particle.

17. The pharmaceutical dosage form of claim 16, the labeling mixture comprises at least 25 unique identifiers, and wherein the at least 25 unique identifiers are selected from a set of at least 50 unique identifiers.

18. The pharmaceutical dosage form of claim 16, wherein the dosage form is a solid oral dosage form, and wherein the labeling composition is associated with the solid oral dosage form by dusting the dosage form with the labeling composition.

19. The pharmaceutical dosage form of claim 16, wherein the labeling mixture comprises a plurality of unique identifiers associated with a carrier particle and wherein the carrier particle is a ferrite bead.

20. The pharmaceutical dosage form of claim 16, wherein the unique identifiers are selected from the group consisting of an oligonucleotide, a carrier particle, a pharmaceutically acceptable excipient, a dye, a peptide, an oligoglycan, a mass tag, and combinations thereof.

21. A method for labeling an item, comprising
a) providing an item;
b) associating the item with a labeling mixture that comprises a combination of at least 25 unique identifiers, wherein the at least 25 unique identifiers are selected from a set of at least 50 unique identifiers, and wherein the labeling mixture is either associated directly with the item or is indirectly associated with the item via a carrier particle; and
c) documenting the combination of at least twenty-five identifiers associated with the item.

22. The method of claim 21, wherein the item is selected from the group consisting of a pharmaceutical dosage form, a dermal patch, a medical device, a certificate of value, and currency.

23. The method of claim 22, wherein the pharmaceutical dosage form is a solid oral dosage form.

24. The method of claim 21, wherein the associating step is by dusting.

25. The method of claim 21, wherein the carrier particle is a ferrite bead.

26. The method of claim 21, wherein the at least 25 unique identifiers are selected from the group consisting of an oligonucleotide, a carrier particle, a pharmaceutically acceptable excipient, a dye, a peptide, an oligoglycan, a mass tag, and combinations thereof.

27. A method of identifying a labeled item comprising:
a) obtaining an item that may have been labeled with an associated labeling mixture;
b) determining the combination of at least twenty-five identifiers, if present; and
c) comparing the determined combination with documentation to identify the source of the item.

28. The method of claim 27, wherein the determining step involves the use of an epifluorescence microscope, a spectrophotometer, a Coulter counter, or a combination thereof.


SEQUENCE LISTING (75 sequences)

SEQ ID NO: 1 - DNA, 6 bp, Artificial Sequence
  Description of Artificial Sequence Synthetic oligonucleotide
aaaaaa

SEQ ID NO: 2 - DNA, 6 bp, Artificial Sequence
  Description of Artificial Sequence Synthetic oligonucleotide
aacaac

SEQ ID NO: 3 - DNA, 6 bp, Artificial Sequence
  Description of Artificial Sequence Synthetic oligonucleotide
aagaag

SEQ ID NO: 4 - DNA, 6 bp, Artificial Sequence
  Description of Artificial Sequence Synthetic oligonucleotide
aataat

SEQ ID NO: 5 - DNA, 6 bp, Artificial Sequence
  Description of Artificial Sequence Synthetic oligonucleotide
acaaca

SEQ ID NO: 6 - DNA, 6 bp, Artificial Sequence
  Description of Artificial Sequence Synthetic oligonucleotide
accacc

SEQ ID NO: 7 - DNA, 6 bp, Artificial Sequence
  Description of Artificial Sequence Synthetic oligonucleotide
acgacg

SEQ ID NO: 8 - DNA, 6 bp, Artificial Sequence
  Description of Artificial Sequence Synthetic oligonucleotide
actact

SEQ ID NO: 9 - DNA, 6 bp, Artificial Sequence
  Description of Artificial Sequence Synthetic oligonucleotide
agaaga

SEQ ID NO: 10 - DNA, 6 bp, Artificial Sequence
  Description of Artificial Sequence Synthetic oligonucleotide
agcagc

SEQ ID NO: 11 - DNA, 6 bp, Artificial Sequence
  Description of Artificial Sequence Synthetic oligonucleotide
aggagg

SEQ ID NO: 12 - DNA, 6 bp, Artificial Sequence
  Description of Artificial Sequence Synthetic oligonucleotide
agtagt

SEQ ID NO: 13 - DNA, 6 bp, Artificial Sequence
  Description of Artificial Sequence Synthetic oligonucleotide
ataata

SEQ ID NO: 14 - DNA, 6 bp, Artificial Sequence
  Description of Artificial Sequence Synthetic oligonucleotide
atcatc

SEQ ID NO: 15 - DNA, 6 bp, Artificial Sequence
  Description of Artificial Sequence Synthetic oligonucleotide
atgatg

SEQ ID NO: 16 - DNA, 6 bp, Artificial Sequence
  Description of Artificial Sequence Synthetic oligonucleotide
attatt

SEQ ID NO: 17 - DNA, 6 bp, Artificial Sequence
  Description of Artificial Sequence Synthetic oligonucleotide
caacaa

SEQ ID NO: 18 - DNA, 6 bp, Artificial Sequence
  Description of Artificial Sequence Synthetic oligonucleotide
caccac

SEQ ID NO: 19 - DNA, 6 bp, Artificial Sequence
  Description of Artificial Sequence Synthetic oligonucleotide
cagcag

SEQ ID NO: 20 - DNA, 6 bp, Artificial Sequence
  Description of Artificial Sequence Synthetic oligonucleotide
catcat

SEQ ID NO: 21 - DNA, 6 bp, Artificial Sequence
  Description of Artificial Sequence Synthetic oligonucleotide
ccacca

SEQ ID NO: 22 - DNA, 6 bp, Artificial Sequence
  Description of Artificial Sequence Synthetic oligonucleotide
cccccc

SEQ ID NO: 23 - DNA, 6 bp, Artificial Sequence
  Description of Artificial Sequence Synthetic oligonucleotide
cctcct

SEQ ID NO: 24 - DNA, 6 bp, Artificial Sequence
  Description of Artificial Sequence Synthetic oligonucleotide
cgacga

SEQ ID NO: 25 - DNA, 6 bp, Artificial Sequence
  Description of Artificial Sequence Synthetic oligonucleotide
cgccgc

SEQ ID NO: 26 - DNA, 6 bp, Artificial Sequence
  Description of Artificial Sequence Synthetic oligonucleotide
cgccgc

SEQ ID NO: 27 - DNA, 6 bp, Artificial Sequence
  Description of Artificial Sequence Synthetic oligonucleotide
cgtcgt

SEQ ID NO: 28 - DNA, 6 bp, Artificial Sequence
  Description of Artificial Sequence Synthetic oligonucleotide
ctacta

SEQ ID NO: 29 - DNA, 6 bp, Artificial Sequence
  Description of Artificial Sequence Synthetic oligonucleotide
ctcctc

SEQ ID NO: 30 - DNA, 6 bp, Artificial Sequence
  Description of Artificial Sequence Synthetic oligonucleotide
ctgctg

SEQ ID NO: 31 - DNA, 6 bp, Artificial Sequence
  Description of Artificial Sequence Synthetic oligonucleotide
cttctt

SEQ ID NO: 32 - DNA, 6 bp, Artificial Sequence
  Description of Artificial Sequence Synthetic oligonucleotide
gaagaa

SEQ ID NO: 33 - DNA, 6 bp, Artificial Sequence
  Description of Artificial Sequence Synthetic oligonucleotide
gacgac

SEQ ID NO: 34 - DNA, 6 bp, Artificial Sequence
  Description of Artificial Sequence Synthetic oligonucleotide
ccgccg

SEQ ID NO: 35 - DNA, 6 bp, Artificial Sequence
  Description of Artificial Sequence Synthetic oligonucleotide
gaggag

SEQ ID NO: 36 - DNA, 6 bp, Artificial Sequence
  Description of Artificial Sequence Synthetic oligonucleotide
gatgat

SEQ ID NO: 37 - DNA, 6 bp, Artificial Sequence
  Description of Artificial Sequence Synthetic oligonucleotide
gcagca

SEQ ID NO: 38 - DNA, 6 bp, Artificial Sequence
  Description of Artificial Sequence Synthetic oligonucleotide
gccgcc

SEQ ID NO: 39 - DNA, 6 bp, Artificial Sequence
  Description of Artificial Sequence Synthetic oligonucleotide
gcggcg

SEQ ID NO: 40 - DNA, 6 bp, Artificial Sequence
  Description of Artificial Sequence Synthetic oligonucleotide
gctgct

SEQ ID NO: 41 - DNA, 6 bp, Artificial Sequence
  Description of Artificial Sequence Synthetic oligonucleotide
ggagga

SEQ ID NO: 42 - DNA, 6 bp, Artificial Sequence
  Description of Artificial Sequence Synthetic oligonucleotide
ggcggc

SEQ ID NO: 43 - DNA, 6 bp, Artificial Sequence
  Description of Artificial Sequence Synthetic oligonucleotide
gggggg

SEQ ID NO: 44 - DNA, 6 bp, Artificial Sequence
  Description of Artificial Sequence Synthetic oligonucleotide
ggtggt

SEQ ID NO: 45 - DNA, 6 bp, Artificial Sequence
  Description of Artificial Sequence Synthetic oligonucleotide
gtagta

SEQ ID NO: 46 - DNA, 6 bp, Artificial Sequence
  Description of Artificial Sequence Synthetic oligonucleotide
gtcgtc

SEQ ID NO: 47 - DNA, 6 bp, Artificial Sequence
  Description of Artificial Sequence Synthetic oligonucleotide
gtggtg

SEQ ID NO: 48 - DNA, 6 bp, Artificial Sequence
  Description of Artificial Sequence Synthetic oligonucleotide
gttgtt

SEQ ID NO: 49 - DNA, 6 bp, Artificial Sequence
  Description of Artificial Sequence Synthetic oligonucleotide
taataa

SEQ ID NO: 50 - DNA, 6 bp, Artificial Sequence
  Description of Artificial Sequence Synthetic oligonucleotide
tactac

SEQ ID NO: 51 - DNA, 6 bp, Artificial Sequence
  Description of Artificial Sequence Synthetic oligonucleotide
tagtag

SEQ ID NO: 52 - DNA, 6 bp, Artificial Sequence
  Description of Artificial Sequence Synthetic oligonucleotide
tattat

SEQ ID NO: 53 - DNA, 6 bp, Artificial Sequence
  Description of Artificial Sequence Synthetic oligonucleotide
tcatca

SEQ ID NO: 54 - DNA, 6 bp, Artificial Sequence
  Description of Artificial Sequence Synthetic oligonucleotide
tcctcc

SEQ ID NO: 55 - DNA, 6 bp, Artificial Sequence
  Description of Artificial Sequence Synthetic oligonucleotide
tcgtcg

SEQ ID NO: 56 - DNA, 6 bp, Artificial Sequence
  Description of Artificial Sequence Synthetic oligonucleotide
tcttct

SEQ ID NO: 57 - DNA, 6 bp, Artificial Sequence
  Description of Artificial Sequence Synthetic oligonucleotide
tgatga

SEQ ID NO: 58 - DNA, 6 bp, Artificial Sequence
  Description of Artificial Sequence Synthetic oligonucleotide
tgctgc

SEQ ID NO: 59 - DNA, 6 bp, Artificial Sequence
  Description of Artificial Sequence Synthetic oligonucleotide
tggtgg

SEQ ID NO: 60 - DNA, 6 bp, Artificial Sequence
  Description of Artificial Sequence Synthetic oligonucleotide
tgttgt

SEQ ID NO: 61 - DNA, 6 bp, Artificial Sequence
  Description of Artificial Sequence Synthetic oligonucleotide
ttatta

SEQ ID NO: 62 - DNA, 6 bp, Artificial Sequence
  Description of Artificial Sequence Synthetic oligonucleotide
ttcttc

SEQ ID NO: 63 - DNA, 6 bp, Artificial Sequence
  Description of Artificial Sequence Synthetic oligonucleotide
ttgttg

SEQ ID NO: 64 - DNA, 6 bp, Artificial Sequence
  Description of Artificial Sequence Synthetic oligonucleotide
tttttt

SEQ ID NO: 65 - DNA, 18 bp, Artificial Sequence
  Description of Artificial Sequence Synthetic oligonucleotide
gtacgtaaaaaagcgcgc

SEQ ID NO: 66 - DNA, 18 bp, Artificial Sequence
  Description of Artificial Sequence Synthetic oligonucleotide
gtacgtaacaacgcgcgc

SEQ ID NO: 67 - DNA, 9 bp, Artificial Sequence
  Description of Artificial Sequence Synthetic oligonucleotide
gcgcgcttt

SEQ ID NO: 68 - DNA, 9 bp, Artificial Sequence
  Description of Artificial Sequence Synthetic oligonucleotide
tttacgtac

SEQ ID NO: 69 - DNA, 9 bp, Artificial Sequence
  Description of Artificial Sequence Synthetic oligonucleotide
gcgcgcgtt

SEQ ID NO: 70 - DNA, 9 bp, Artificial Sequence
  Description of Artificial Sequence Synthetic oligonucleotide
gttacgtac

SEQ ID NO: 71 - DNA, 21 bp, Artificial Sequence
  Description of Artificial Sequence Synthetic oligonucleotide
gccgatcgaatgagagcgcgc

SEQ ID NO: 72 - DNA, 9 bp, Artificial Sequence
  Description of Artificial Sequence Synthetic oligonucleotide
ttttttacg

SEQ ID NO: 73 - DNA, 9 bp, Artificial Sequence
  Description of Artificial Sequence Synthetic oligonucleotide
gttgttacg

SEQ ID NO: 74 - DNA, 34 bp, Artificial Sequence
  Description of Artificial Sequence Synthetic oligonucleotide
gtacgtaacaacgcgcgcctctctaagctagccg

SEQ ID NO: 75 - DNA, 31 bp, Artificial Sequence
  Description of Artificial Sequence Synthetic oligonucleotide
cggctagcttagagaggcgcgcgttgttacg